(19) United States
(12) Patent Application Publication
(10) Pub. No.: US 2009/0098079 A1
(43) Pub. Date: Apr. 16, 2009

(54) PRODUCT RELEASE SYSTEM FOR ATOMIZING COSMETIC HAIR COMPOSITIONS CONTAINING CATIONIC POLYMERS

(75) Inventors: Hartmut Schiemann, Hunfeld (DE); Thomas Krause, Darmstadt (DE); Michael Franzke, Robdorf (DE); Dirk Weber, Marly (CH); Monika Moenks, Schmitten (CH); Jan Baumeister, Farvagny-le-Grand (CH); Ellen Florig, Grasellenbach (DE)

Correspondence Address: THE PROCTER & GAMBLE COMPANY;Global Legal Department - IP, Sycamore Building - 4th Floor, 299 East Sixth Street, CINCINNATI, OH, 45202, US

(73) Assignee: The Procter & Gamble Company, Cincinnati, OH (US)

(21) Appl. No.: 12/335,592

(22) Filed: Dec. 16, 2008

Related U.S. Application Data
(63) Continuation of application No. PCT/US2006/023923, filed on Jun. 20, 2006.

(30) Foreign Application Priority Data
Jun. 20, 2005 (DE) 102005028382.9

(51) Int. Cl. A61K 8/81; A61K 8/89; A61Q 5/00

(52) U.S. Cl. 424/70.11; 424/70.15; 424/70.122

(57) ABSTRACT

A product release system for atomizing cosmetic hair compositions is described, which has (a) pressure-resistant packaging, (b) a capillary-containing spray head, and (c) a propellant-containing cosmetic composition, which contains at least one film-forming, hair-setting, or hair-conditioning cationic polymer. The atomization is done using the capillary. The capillary preferably has a diameter of 0.1 to 1 mm and a length of 5 to 100 mm. The spray rate is preferably 0.01 to 5 g/s. The composition can particularly be a gel, wax, or emulsion.

The object of the present invention is a product release system for atomizing cosmetic compositions, which has pressure-resistant packaging, a capillary-containing spray head, and a propellant-containing cosmetic composition, and wherein the composition contains at least one film-forming, hair-setting, or hair-conditioning cationic polymer. The object of the invention is thus a corresponding method for hair treatment.

Many cosmetic hair products contain cationic polymers as hair-setting or hair-conditioning ingredients. In the form of foams, cationic polymers provide the hair with good combing properties after application in wet hair and effect good setting with a good hold in dry hair. In shampoos, permanent wave solutions, fixing products, and hair colors, cationic polymers are used to provide good combing properties in wet hair and good hold properties in dry hair. They provide hairstyle stability and increase hair volume in dry hair. However, cationic polymers have not been widely used up to now in higher-viscosity products such as gels, waxes, hairdressing creams, or treatments, because the composition as well as the product consistency and the resulting more difficult application meant that the advantages of the cationic polymers couldn't be fully realized on the hair.

A process for atomizing liquid is known from WO 03/051523 A1 with which the spray is formed using a capillary. Only the application with respect to atomizing liquid compositions is described. A fixture for atomizing liquid products is described in WO 03/051522 A2, wherein the spray is formed using a capillary. Only the use of liquid compositions for atomizing, which can also be highly viscous, is described, wherein, however, 5,000 mPa s is mentioned as the maximum sprayable viscosity.

There is a need for products that will realize the advantageous hair-setting and hair-conditioning properties of cationic polymers even in application forms such as hair waxes, styling gels, hairdressing creams, or creamy treatments and that will combine the advantageous properties with these forms of application. The object of the invention, in particular, was to further improve the distributability on hair, the volume effect, the setting effect and/or hairstyle stability, the combing properties, the hair shine, and/or the holding properties of conventional hair treatment compositions, which were previously unavailable as a spray, and/or the hair treated with said composition.

The object of the invention is a product release system for atomizing cosmetic compositions. The product release system has the following features:

(a) pressure-resistant packaging;

(b) a spray head containing a capillary; and

(c) a propellant-containing cosmetic composition,

wherein atomization is done using the capillary and the composition contains at least one film-forming, hair-setting, or hair-conditioning cationic polymer.

The term “atomize” is understood to mean the release of the product in the form of dissipated particles. The dissipated particles can have varying shapes, consistencies, and sizes. The properties of the atomized particles can include everything from fine aerosol atomized spray to liquid drops, snow-like drops, solid spray flakes, and spray foam.

The quantities of ingredients (e.g., wt. %) indicated in the following are each based on the basic composition without propellant unless explicitly indicated otherwise. The quantities of the propellant are based on the total composition including propellant.

The composition is preferably non-liquid at 25° C. and/or has a viscosity greater than 5,000 mPa s (measured with a HAAKE VT-550 Rheometer, SV-DIN test body at a temperature of 25° C. and a shear speed of 12.9 s⁻¹).

The properties of the compositions to be used according to the invention that are related to consistency are based on the base composition without propellant (unless explicitly indicated otherwise). Non-liquid compositions in terms of the invention are particularly non-flow-capable compositions, which, for example, can be determined due to the fact that they will not flow off of a glass surface tilted at 45° at a temperature of 25° C. Gel compositions are characterized in that the memory module G′ is larger than the loss module G″ at 25° C. with oscillographic measurements in the typical measurement range (0.01 to 40 Hz).

The viscosity of the composition to be used is preferably greater than 5,000 up to 100,000, and especially preferably 10,000 to 50,000 mPa s, or very especially preferably 25,000 to 35,000 mPa s, measured with a HAAKE VT-550 Rheometer, SV-DIN test body at a temperature of 25° C. and a shear speed of 12.9 s⁻¹.

Aerosol spray cans constructed of metal or plastic can be used as the pressure-resistant packaging. Preferred metals are tin plates and aluminum, while the preferred plastic is polyethylene terephthalate.

Suitable spray systems with capillary-containing spray heads, with which the spray is formed using a capillary, are described in WO 03/051523 A1 and in WO 03/051522 A2. The capillaries preferably have a diameter of 0.1 to 1 mm, or particularly 0.2 to 0.6 mm, and a length that is preferably 5 to 100 mm, or particularly 5 to 50 mm. The spray principle is also described in Aerosol Europe, vol. 13, no. 1-2005, pages 6-11. The spray system is based on the principle of capillary atomization. The conventional swirl nozzle as well as, if necessary, the uptake tube are replaced by capillaries. The energy-consuming and propellant-intensive swirling of the content of the can and the required strong dilution of the product with solvents is not necessary as compared to conventional spray systems. Even if only a small quantity of propellant is used, the product rises upward on the wall of the uptake tube capillary and is propelled, after the valve in the (wider) capillary of the spray head, in the direction of the exit opening. In this manner, small drops from the flowing propellant are torn from the surface of the liquid and continue to flow as aerosol. Since there is no swirl chamber to inhibit the flow of the product nor any atomizing nozzle available, the energy in the system can be used much more efficiently to create the desired spray. The spray rate can be adjusted via the selection of the capillary geometry in conjunction with the interior pressure created by the propellant or a propellant mixture. Preferred spray rates are 0.01058 to 0.5 g/s, particularly 0.1 to 0.3 g/s. The size of the spray drops created with the atomization can be adjusted via the selection of the capillary geometry in conjunction with the interior pressure or the viscosity of the composition. Suitable capillary atomization systems can be obtained in a product called TRUSPRAY® from Boehringer Ingelheim microParts GmbH.

The preferred drop size distributions are those with which the dv(50) value is a maximum of 100 μm, e.g., of from 50 to 100 μm, with a maximum of 90 μm being especially preferred, e.g., of from 70 to 90 μm, and/or with which the dv(90) value is a maximum of 160 μm, e.g., of from 90 to 160 μm, with a maximum of 150 μm being especially preferred, e.g., of from 115 to 150 μm. The dv(50) or dv(90) values provide the maximum diameter that 50% or 90% of all droplets have. The drop size distribution can, for example, be determined with the help of a particle measurement unit based on laser beam diffraction, e.g., a Malvern particle sizer measuring device. Compositions that form a snow-like consistency, flakes, or foam (spray foam) upon exiting the capillary spray system are also preferred.

The propellant to be used can be selected from lower alkanes, particularly C3 to C5 hydrocarbons such as, for example, n-butane, i-butane, and propane, or also mixtures thereof, as well as dimethylethers or fluorine hydrocarbons such as F 152a (1,1-difluoroethane) or F 134 (tetrafluoroethane) as well as other gaseous propellants present with the pressures considered, such as, for example, N₂, N₂O, and CO₂ as well as mixtures of the aforementioned propellants. The propellant is preferably selected from propane, n-butane, isobutane, dimethylether, fluorinated hydrocarbons, and mixtures thereof. The content of propellant is, in addition, preferably 15 to 85 wt. %, with 25 to 75 wt. % being especially preferred.

The composition contains cosmetically acceptable solvents, preferably an aqueous, alcoholic, or aqueous alcoholic medium. The lower alcohols with 1 to 4 C atoms, such as ethanol and isopropanol, can be contained as alcohols, particularly those typically used for cosmetic purposes. The composition can be in a pH range of 2.0 to 9.5. A pH range of 4 to 8 is particular preferred, providing no special application forms require other pH values. As additional co-solvents, organic solvents or a mixture of solvents with a boiling point of less than 400° C. can be contained in a quantity of from 0.1 to 15 wt. % or preferably of from 1 to 10 wt. %. Unbranched or branched hydrocarbons such as n-pentane, hexane, isopentane, and cyclic hydrocarbons such as cyclopentane and cyclohexane are particularly suitable as additional co-solvents. These volatile hydrocarbons can also be used as propellants. Other, especially preferred water-soluble solvents are glycerol, ethylene glycol, and propylene glycol in a quantity of up to 30 wt. %.

The product release system according to the invention can be used for hair treatment. The compositions can be agents for the care of hair such as, for example, hair-repair products or hair rinses, which, for example, can be applied as leave-on or rinse-off products; agents for the temporary reshaping and/or stabilizing of the hairstyle (styling agent), for example hair sprays, hair lacquers, hair gels, hair waxes, styling creams, etc.; permanent, semipermanent, or temporary hair colorants, for example oxidative hair colorants or nonoxidative hair tinting agents or hair bleaching agents; permanent hair restructuring agents, for example in the form of a mildly alkaline or acidic permanent wave or hair straightening agents containing a reducing agent, or in the form of permanent wave fixing agents containing an oxidizing agent.

The cationic polymers are contained in the composition to be used according to the present invention in a quantity that is preferably 0.01 to 20 wt. % or 0.05 to 10 wt. %, with 0.1 to 5 wt. % being particularly preferred. The polymers can be synthetic or natural polymers. The polymers are hair-setting and/or hair-conditioning polymers that preferably form a film as well. Natural polymers are understood to also include chemically modified polymers of natural origin. Hair-setting polymers are understood to be those capable of exhibiting a setting effect on the hair or a stabilizing effect on the hairstyle when used in a 0.01 to 5% aqueous, alcoholic, or aqueous alcoholic solution or dispersion, e.g., those that increase curl retention with respect to a water wave, especially those for which the “Hair Fixatives” function is indicated in the International Cosmetic Ingredient Dictionary and Handbook, 10th edition, 2004. Hair-conditioning polymers are understood to be those capable of exhibiting a hair-conditioning or conditioning effect on the hair when used in a 0.01 to 5% aqueous, alcoholic, or aqueous alcoholic solution or dispersion, e.g., those that improve the combing ability or increase shine, especially those for which the “Hair Conditioning Agents” function is indicated in the International Cosmetic Ingredient Dictionary and Handbook, 10th edition, 2004. Film-forming polymers are understood to be those capable of depositing a polymer film on the hair after drying when used in a 0.01 to 5% aqueous, alcoholic, or aqueous alcoholic solution or dispersion, especially those for which the “Film Formers” function is indicated in the International Cosmetic Ingredient Dictionary and Handbook, 10th edition, 2004. The polymers can also simultaneously have two or three of the properties known as “film-forming,” “hair-setting,” and “hair-conditioning.”

Cationic polymers are polymers with cationic groups or with amine groups, particularly primary, secondary, tertiary, or quaternary amine groups. The cationic charge density will preferably be 1 to 7 meq/g.

Suitable synthetic cationic polymers are homo- or copolymers consisting of at least one of the following monomers:dialkylaminoalkyl acrylate, dialkylaminoalkyl methacrylate, monoalkylaminoalkyl acrylate, and monoalkyl aminoalkyl methacrylate, trialkyl methacryloxyalkyl ammonium, trialkyl acryloxyalkyl ammonium, dialkyl diallyl ammonium, and quaternary vinyl ammonium monomers with cyclic groups containing cationic nitrogens.

Suitable cationic polymers preferably contain quaternary amino groups. Cationic polymers can be homo- or copolymers, where the quaternary nitrogen groups are contained either in the polymer chain or preferably as substituents on one or more of the monomers. The monomers containing ammonium groups can be copolymerized with non-cationic monomers. Suitable cationic monomer are unsaturated compounds that can undergo radical polymerization, which bear at least one cationic group, especially ammonium-substituted vinyl monomers such as, for example, trialkylmethacryloxyalkylammonium, trialkylacryloxyalkylammonium, dialkyldiallylammonium and quaternary vinylammonium monomers with cyclic, cationic nitrogen-containing groups such as pyridinium, imidazolium or quaternary pyrrolidones, e.g., alkylvinylimidazolium, alkylvinylpyridinium, or alkylvinylpyrrolidone salts. The alkyl groups of these monomers are preferably lower alkyl groups such as, for example, C1 to C7 alkyl groups, and especially preferred are C1 to C3 alkyl groups.

The monomers containing ammonium groups can be copolymerized with non-cationic monomers. Suitable comonomers are, for example, acrylamide, methacrylamide, alkyl- and dialkylacrylamide, alkyl- and dialkylmethacrylamide, alkyl acrylate, alkyl methacrylate, vinylcaprolactone, vinylcaprolactam, vinylpyrrolidone, vinyl esters, for example vinyl acetate, vinyl alcohol, propylene glycol or ethylene glycol, wherein the alkyl groups of these monomers are preferably C1 to C7 alkyl groups, and especially preferred are C1 to C3 alkyl groups.

Suitable polymers with quaternary amino groups are, for example, those described in the CTFA Cosmetic Ingredient Dictionary under the designations Polyquaternium such as methylvinylimidazolium chloride/vinylpyrrolidone copolymer (Polyquatemium-16) or quaternized vinylpyrrolidone/dimethylaminoethyl methacrylate copolymer (Polyquaternium-11) as well as quaternary silicone polymers or silicone oligomers such as, for example, silicone polymers with quaternary end groups (Quaternium-80).

Preferred cationic polymers of synthetic origin:

poly(dimethyldiallyl ammonium chloride); copolymers from acrylamide and dimethyldiallyl ammonium chloride; quaternary ammonium polymers, formed by the reaction of diethyl sulfate with a copolymer from vinylpyrrolidone and dimethylaminoethyl methacrylate, especially vinylpyrrolidone/dimethylaminoethyl methacrylate methosulfate copolymer (e.g., Gafquat® 755 N, Gafquat® 734); quaternary ammonium polymers from methylvinylimidazolium chloride and vinylpyrrolidone (e.g., LUVIQUAT® HM 550); Polyquatemium-35; Polyquatemium-57; polymers from trimethylammonium ethyl methacrylate chloride; terpolymers from dimethyldiallyl ammonium chloride, sodium acrylate and acrylamide (e.g., Merquat® Plus 3300); copolymers from vinylpyrrolidone, dimethylaminopropyl methacrylamide and methacryloylaminopropyllauryldimethylammonium chloride; terpolymers from vinylpyrrolidone, dimethylaminoethyl methacrylate and vinylcaprolactam (e.g., Gaffix® VC 713); vinylpyrrolidone/methacrylamidopropyltrimethylammonium chloride copolymers (e.g., Gafquat® HS 100); copolymers from vinylpyrrolidone and dimethylaminoethyl methacrylate; copolymers from vinylpyrrolidone, vinylcaprolactam and dimethylaminopropylacrylamide; poly- or oligoesters formed from at least one first type of monomer, that is selected from hydroxyacids substituted with at least one quaternary ammonium group; dimethylpolysiloxane substituted with quaternary ammonium groups in the terminal positions.

Suitable cationic polymers that are derived from natural polymers are especially cationic derivatives of polysaccharides, for example, cationic derivatives of cellulose, starch or guar. Furthermore, chitosan and chitosan derivatives are also suitable. Cationic polysaccharides are, for example, represented by the general formula

G-O—B—N⁺R^{a}R^{b}R^{c}X⁻

G is an anhydroglucose residue, for example, starch or cellulose anhydroglucose;

B is a divalent linking group, for example alkylene, oxyalkylene, polyoxyalkylene or hydroxyalkylene;

R^{a}, R^{b}, and R^{c}, independently from one another, are alkyl, aryl, alkylaryl, arylalkyl, alkoxyalkyl, or alkoxyaryl, any of which can have up to 18 C atoms, wherein the total number of C atoms in R^{a}, R^{b}, and R^{c} is preferably a maximum of 20;

X is a conventional counter-anion, for example, a halide, acetate, phosphate, nitrate, or alkyl sulfate, preferably a chloride. Cationic celluloses are, for example, those with the INCI names Polyquaternium-4, Polyquaternium-10, or Polyquaternium-24. A suitable cationic guar derivative has, for example, the INCI designation Guar Hydroxypropyltrimonium Chloride.

Especially preferred cationically-active substances are chitosan, chitosan salts and chitosan derivatives. Chitosans that can be used according to the invention can be fully or partially deacetylated chitins. By way of example, the molecular weight can be distributed over a broad range, from 20,000 to about 5 million g/mol, for example from 30,000 to 70,000 g/mol. However, the molecular weight will preferably lie above 100,000 g/mol, and especially preferred from 200,000 to 700,000 g/mol. The degree of deacetylation is preferably from 10 to 99%, and especially preferably from 60 to 99%. A preferred chitosan salt is chitosonium pyrrolidone carboxylate, e.g., Kytamer® PC with a molecular weight of from about 200,000 to 300,000 g/mol and a degree of deacetylation of from 70 to 85%. Chitosan derivatives that can be considered include quaternized, alkylated or hydroxyalkylated derivatives, e.g., hydroxyethyl, hydroxypropyl or hydroxybutyl chitosan. The chitosans or chitosan derivatives are preferably present in their neutralized or partially neutralized form. The degree of neutralization will be preferably at least 50%, especially preferably between 70 and 100%, as calculated on the basis of the number of free base groups. For the neutralization agent, in principle any cosmetically compatible inorganic or organic acids can be used such as, for example, formic acid, tartaric acid, malic acid, lactic acid, citric acid, pyrrolidone carboxylic acid, hydrochloric acid and others, of which pyrrolidone carboxylic acid is especially preferred.

Preferred cationic polymers derived from natural sources:

cationic cellulose derivatives from hydroxyethyl cellulose and diallyldimethyl ammonium chloride; cationic cellulose deviates from hydroxyethyl cellulose and trimethylammonium-substituted epoxide; chitosan and its salts; hydroxyalkyl chitosans and their salts; alkylhydroxyalkyl chitosans and their salts; N-hydroxyalkylchitosan alkyl ethers.

In one embodiment, the composition to be used according to the invention is a gel and contains at least one thickener or gel-former preferably in a quantity of from 0.01 to 20 wt. % or of from 0.1 to 10 wt. %, of from 0.5 to 8 wt. %, or especially preferably of from 1 to 5 wt. %. Materials for which the function “Viscosity Increasing Agent” is indicated in the International Cosmetic Ingredient Dictionary and Handbook, 10th edition, 2004 are essentially suitable. The thickener or gel-former is preferably a thickening polymer and is especially preferably selected from copolymers consisting of at least one first type of monomer, which is selected from acrylic acid and methacrylic acid, and at least one second type of monomer, which is selected from esters of acrylic acid and ethoxylated fatty alcohol; crosslinked polyacrylic acid; crosslinked copolymers consisting of at least one first type of monomer, which is selected from acrylic acid and methacrylic acid, and at least one second type of monomer, which is selected from esters of acrylic acid with C10 to C30 alcohols; copolymers consisting of at least one first type of monomer, which is selected from acrylic acid and methacrylic acid, and at least one second type of monomer, which is selected from esters of itaconic acid and ethoxylated fatty alcohol; copolymers consisting of at least one type of monomer, which is selected from acrylic acid and methacrylic acid, at least one second type of monomer, which is selected from esters of itaconic acid and ethoxylated C10 to C30 alcohol, and a third type of monomer, which is selected from C1 to C4 aminoalkyl acrylates; copolymers consisting of two or more monomers, which are selected from acrylic acid, methacrylic acid, acrylic acid esters and methacrylic acid esters; copolymers consisting of vinyl pyrrolidone and ammonium acryloyl dimethyltaurate; copolymers consisting of ammonium acryloyl dimethyltaurate and monomers selected from esters of methacrylic acid and ethoxylated fatty alcohols; hydroxyethyl cellulose; hydroxypropyl cellulose; hydroxypropyl guar; glyceryl polyacrylate; glyceryl polymethacrylate; copolymers consisting of at least one C2, C3, or C4 alkylene and styrene; polyurethane; hydroxypropyl starch phosphate; polyacrylamide; copolymers crosslinked with decadiene consisting of maleic acid anhydride and methyl vinyl ether; locust bean gum; guar gum; xanthan; dehydroxanthan; carrageenan; karaya gum; hydrolyzed corn starch; copolymers consisting of polyethylene oxide, fatty alcohols, and saturated methylene diphenyl diisocyanate (e.g., PEG-150/stearyl alcohol/SMDI copolymer).

In an additional embodiment, the composition is waxy and contains at least one wax that is solid at 25° C. preferably in a quantity of from 10 to 80 wt. %, particularly of from 20 to 60 wt. %, or of from 25 to 50 wt. %, as well as, if necessary, other water-insoluble materials that are liquid at room temperature. The waxy consistency is preferably characterized in that the needle penetration number (unit of measurement 0.1 mm), test weight 100 g, testing time 5 s, test temperature 25° C. (according to DIN 51 579) preferably ranges from 2 to 70, or particularly from 3 to 40, and/or that the composition can be melted and has a solidification point that is greater than 25° C., or is preferably in a range of from 30 to 70° C., or especially preferably in a range of from 40 to 55° C.

Principally any wax that is known in the prior art can be used as a wax or waxy material. These waxes include animal, vegetable, mineral, and synthetic waxes, microcrystalline waxes, macrocrystalline waxes, solid paraffins, petroleum jelly, Vaseline, ozocerite, montan wax, Fischer-Tropsch wax, polyolefin waxes, e.g., polybutene, beeswax, wool wax, and its derivatives such as, for example, wool wax alcohols, candelilla wax, olive wax, carnauba wax, Japan wax, apple wax, hydrogenated fats, fatty acid esters, fatty acid glycerides with a solidification point greater than 40° C., silicone waxes or hydrophilic waxes such as, for example, high-molecular-weight polyethylene glycol waxes with a molecular weight of from 800 to 20,000, preferably of from 2,000 to 10,000 g/mol. The waxes or waxy materials have a solidification point greater than 25° C., or preferably greater than 40° C. or 55° C. The needle penetration number (0.1 mm), 100 g, 5 s, 25° C. (according to DIN 51 579) preferably lies in the range of from 2 to 70, or especially 3 to 40.

In another embodiment, the composition is emulsion-like, wherein the consistency is preferably creamy. The emulsion can be a water-in-oil emulsion, an oil-in-water emulsion, a microemulsion, or a higher emulsion. In addition to water, preferably at least one hydrophobic oil that is liquid at room temperature (25° C.) as well as at least one emulsifier is contained. The oil content is preferably of from 1 to 20 wt. %, or particularly of from 2 to 10 wt. %. The emulsifier content is preferably of from 0.01 to 30 wt. %, or particularly of from 0.1 to 20 wt. % or of from 0.5 to 10 wt. %.

Suitable liquid, hydrophobic oils have a melting point of less than 25° C. and a boiling point of preferably greater than 250° C., or particularly greater than 300° C. Volatile oils can also be used. In principle, any oil generally known to a person skilled in the art can be used. Suitable oils are vegetable or animal oils, mineral oils (liquid paraffin), silicone oils or their mixtures. Hydrocarbon oils, e.g., paraffin or isoparaffin oils, squalane, oils from fatty acids and polyols, especially triglycerides, are suitable. Suitable vegetable oils are, for example, sunflower oil, coconut oil, castor oil, lanolin oil, jojoba oil, corn oil, soy oil.

Suitable emulsifiers can include nonionic, anionic, cationic, or zwitterionic surfactants. Suitable nonionic surfactants are, for example,
ethoxylated fatty alcohols, fatty acids, fatty acid glycerides, or alkyl phenols, especially addition products of 2 to 30 mol ethylene oxide and/or 1 to 5 mol propylene oxide to C8 to C22 fatty alcohols, to C12 to C22 fatty acids, or to alkyl phenols with 8 to 15 C atoms in the alkyl group;
C12 to C22 fatty acid mono- and diesters of addition products of 1 to 30 mol ethylene oxide to glycerol;
addition products of 5 to 60 mol ethylene oxide to castor oil or hydrogenated castor oil;
fatty acid sugar esters, especially esters from saccharose and one or two C8 to C22 fatty acids, INCI: Sucrose Cocoate, Sucrose Dilaurate, Sucrose Distearate, Sucrose Laurate, Sucrose Myristate, Sucrose Oleate, Sucrose Palmitate, Sucrose Ricinoleate, Sucrose Stearate;
esters from sorbitan and one, two or three C8 to C22 fatty acids and a degree of ethoxylation of 4 to 20;
polyglyceryl fatty acid esters, especially from one, two or more C8 to C22 fatty acids and polyglycerol with preferably 2 to 20 glyceryl units;
alkylglucosides, alkyloligoglucosides, and alkylpolyglucoside with C8 to C22 alkyl groups, e.g., decyl glucoside or lauryl glucoside.

Suitable anionic surfactants are, for example, salts and esters of carboxylic acids, alkyl ether sulfates and alkyl sulfates, fatty alcohol ether sulfates, sulfonic acids and their salts (e.g., sulfosuccinates or fatty acid isethienates), phosphoric acid esters and their salts, acylamino acids and their salts. A comprehensive description of these anionic surfactants is found in the publication “FIEDLER—Lexikon der Hilfsstoffe” [FIEDLER—Dictionary of Adjuvants], volume 1, fifth edition (2002), pages 97 to 102, to which expressed reference is made. Preferred surfactants are mono-, di-, and/or triesters of phosphoric acid with addition products of from 2 to 30 mol ethylene oxide to C8 to C22 fatty alcohols.

Suitable amphoteric surfactants are, for example, derivatives of aliphatic quaternary ammonium, phosphonium and sulfonium compounds of the formula

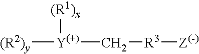

wherein R¹ represents a straight-chain or branched-chain alkyl, alkenyl, or hydroxyalkyl group with 8 to 18 C atoms and 0 to about 10 ethylene oxide units and 0 to 1 glycerol units; Y is an N-, P-, or S-containing group; R² is an alkyl or monohydroxyalkyl group with 1 to 3 C atoms; the total of x+y equals 2 if Y is a sulfur atom, and the total of x+y equals 3 if Y is a nitrogen atom or a phosphorus atom; R³ is an alkylene or hydroxyalkylene group with 1 to 4 C atoms, and Z⁽⁻⁾ represents a carboxylate, sulfate, phosphonate, or phosphate group. Other amphoteric surfactants such as betaines are also suitable. Examples of betaines include C8 to C18 alkylbetaines such as cocodimethylcarboxymethylbetaine, lauryldimethylcarboxymethylbetaine, lauryldimethyl-alpha-carboxyethylbetaine, cetyldimethylcarboxymethylbetaine, oleyldimethylgammacarboxypropylbetaine, and lauryl-bis-(2-hydroxypropyl)-alpha-carboxyethylbetaine; C8 to C18 sulfobetaines such as cocodimethylsulfopropylbetaine, stearyldimethylsulfopropylbetaine, lauryldimethylsulfoethylbetaine, lauryl-bis-(2-hydroxyethyl)sulfopropylbetaine; the carboxyl derivatives of imidazole, C8 to C18 alkyldimethylammonium acetate, C8 to C18 alkyldimethylcarbonylmethylammonium salts, as well as C8 to C18 fatty acid alkylamidobetaines such as, for example, coconut fatty acid amidopropylbetaine and N-coconut fatty acid amidoethyl-N-[2-(carboxymethoxy)ethyl]glycerol (CTFA name: Cocoamphocarboxyglycinate).

Suitable cationic surfactants contain amino groups or quaternized hydrophilic ammonium groups that carry a positive charge in solution and can be represented by the general formula

N⁽⁺⁾R¹R²R³R⁴X⁽⁻⁾

wherein R¹ to R⁴, independently from one another, stand for aliphatic groups, aromatic groups, alkoxy groups, polyoxyalkylene groups, alkylamido groups, hydroxyalkyl groups, aryl groups, or alkaryl groups with 1 to 22 C atoms, wherein at least one radical has at least 6, preferably at least 8, C atoms and X⁻ represents an anion, for example a halide, acetate, phosphate, nitrate, or alkyl sulfate, but preferably a chloride. In addition to the carbon atoms and the hydrogen atoms, the aliphatic groups can also contain cross-compounds, or other groups, such as, for example, additional amino groups. Examples of suitable cationic surfactants are the chlorides or bromides of alkyldimethylbenzylammonium salts, alkyltrimethylammonium salts, e.g., cetyltrimethylammonium chloride or bromide, tetradecyltrimethylammonium chloride or bromide, alkyldimethylhydroxyethylammonium chlorides or bromides, dialkyldimethylammonium chlorides or bromides, alkylpyridinium salts, for example lauryl- or cetylpyridinium chloride, alkylamidoethyltrimethylammonium ether sulfates as well as compounds with cationic character such as amine oxides, e.g., alkylmethylamine oxides or alkylaminoethyldimethylamine oxides. Especially preferred are C8-22 alkyldimethylbenzylammonium compounds, C8-22 alkyltrimethylammonium compounds, especially cetyltrimethylammonium chloride, C8-22 alkyldimethylhydroxyethylammonium compounds, di-(C8-22 alkyl)-dimethylammonium compounds, C8-22 alkylpyridinium salts, C8-22 alkylamidoethyltrimethylammonium ether sulfates, C8-22 alkylmethylamine oxides, and C8-22 alkylaminoethyldimethylamine oxides.

The cosmetic composition to be used according to the present invention can also contain at least one additional active cosmetic ingredient or additive for the hair or skin/scalp. This active ingredient or additive can, for example, be selected from hair-conditioning materials, hair-setting materials, silicone compounds, photoprotective materials, preservatives, pigments, direct-penetrating hair dyes, particle-shaped materials, oxidizing agents, reducing agents, and oxidative hair colorant precursor products. The active ingredients and additives, depending on the type and intended use, are preferably contained in a quantity of from 0.01 to 20 wt. %, or particularly of from 0.05 to 10 wt. %, or of from 0.1 to 5 wt. %.

In one embodiment, the agent according to the invention, as a hair-conditioning or hair-setting additive, contains at least one polymer with anionic groups or groups that can be anionized preferably in a quantity of from 0.01 to 20 wt. % or of from 0.05 to 10 wt. %, with 0.1 to 5 wt. % being particularly preferred. Groups that can be anionized are understood to be acid groups such as, for example, carboxylic acid, sulfonic acid, or phosphoric acid groups that can be deprotonated using typical bases such as, for example, organic amines or alkali- or alkaline earth hydroxides. The anionic polymers can be partially or completely neutralized with an alkaline neutralizing agent. Such types of agents in which the acidic groups are neutralized in the polymer to 50 to 100%, or especially preferably to 70-100%, are preferred. Organic or inorganic bases can be used as the neutralizing agent. Particular examples of bases are amino alkanols such as, for example, aminomethylpropanol (AMP), triethanolamine or monoethanolamine, and also ammonia, NaOH, and KOH among others.

The anionic polymer can be a homo- or copolymer with acid group-containing monomer units derived from natural or synthetic sources, which, if necessary, can be polymerized with comonomers that contain no acid groups. Among the acid groups that can be considered are sulfonic acid, phosphoric acid, and carboxylic acid groups, of which the carboxylic acid groups are preferred. Suitable acid group-containing monomers are, for example, acrylic acid, methacrylic acid, crotonic acid, maleic acid, and maleic anhydride, maleic acid monoesters, especially the C1 to C7 alkyl monoesters of maleic acid, as well as aldehydrocarboxylic acids or ketocarboxylic acids. Comonomers that are not substituted with acid groups are, for example, acrylamide, methacrylamide, alkyl- and dialkylacrylamide, alkyl and dialkylmethacrylamide, alkyl acrylate, alkyl methacrylate, vinylcaprolactone, vinylpyrrolidone, vinyl ester, vinyl alcohol, propylene glycol or ethylene glycol, amine-substituted vinyl monomers such as, for example, dialkylaminoalkyl acrylate, dialkylaminoalkyl methacrylate, monoalkylaminoalkyl acrylate, and monoalkylaminoalkyl methacrylate, wherein the alkyl groups of these monomers are preferably C1 to C7 alkyl groups, with C1 to C-alkyl groups being especially preferred.

Suitable polymers with acid groups are especially homopolymers of acrylic acid or methacrylic acid, copolymers of acrylic acid or methacrylic acid with monomers selected from acrylic acid or methacrylic acid esters, acrylamides, methacrylamides and vinylpyrrolidone, homopolymers of crotonic acid as well as copolymers of crotonic acid with monomers selected from vinyl esters, acrylic acid or methacrylic acid esters, acrylamides and methacrylamides that are uncrosslinked or crosslinked with polyfunctional agents. A suitable natural polymer is, for example, shellac.

Preferred polymers with acid groups are:

Terpolymers from acrylic acid, alkyl acrylate, and N-alkylacrylamide (INCI designation: Acrylate/Acrylamide Copolymer), especially terpolymers from acrylic acid, ethyl acrylate and N-tert-butylacrylamide; crosslinked or uncrosslinked vinyl acetate/crotonic acid copolymers (INCI designation: VA/Crotonate Copolymer); copolymers from one or more C1 to C5 alkyl acrylates, especially C2 to C4 alkyl acrylates and at least one monomer selected from acrylic acid or methacrylic acid (INCI designation: Acrylate Copolymer), e.g., terpolymers from tert-butyl acrylate, ethyl acrylate and methacrylic acid; sodium polystyrenesulfonate; vinylacetate/crotonic acid/vinyl alkanoate copolymers, for example, copolymers from vinyl acetate, crotonic acid and vinyl propionate; copolymers from vinyl acetate, crotonic acid and vinyl neodecanoate (INCI designations: VA/Crotonate/Vinyl Propionate Copolymer, VA/Crotonate/Vinyl Neodecanoate Copolymer); aminomethylpropanol acrylate copolymers; copolymers from vinylpyrrolidone and at least one further monomer selected from acrylic acid and methacrylic acid as well as, if necessary, acrylic acid esters and methacrylic acid esters; copolymers from methyl vinyl ether and maleic acid monoalkylesters (INCI designations: Ethyl Ester of PVM/MA Copolymer, Butyl Ester of PVM/MA Copolymer); aminomethylpropanol salts of copolymers from allyl methacrylate and at least one further monomer selected from acrylic acid, and methacrylic acid as well as, if necessary, acrylic acid esters and methacrylic acid esters; crosslinked copolymers from ethyl acrylate and methacrylic acid; copolymers from vinyl acetate, mono-n-butyl maleate and isobornyl acrylate; copolymers from two or more monomers selected from acrylic acid and methacrylic acid as well as, if necessary, acrylic acid esters and methacrylic acid esters; copolymers from octylacrylamide and at least one monomer selected from acrylic acid and methacrylic acid as well as, if necessary, acrylic acid esters and methacrylic acid esters; polyesters from diglycol, cyclohexanedimethanol, isophthalic acid and sulfoisophthalic acid, wherein the alkyl groups of the aforementioned polymers as a rule preferably possess 1, 2, 3, or 4 C atoms.

In one embodiment, the agent according to the invention, as a hair-conditioning or hair-setting additive, contains at least one zwitterionic and/or amphoteric polymer preferably in a quantity of from 0.01 to 20 wt. % or of from 0.05 to 10 wt. %, or especially preferably of from 0.1 to 5 wt. %. Zwitterionic polymers simultaneously have at least one anionic and at least one cationic charge. Amphoteric polymers exhibit at least one acidic group (e.g., carboxylic acid or sulfonic acid group) and at least one alkaline group (e.g., amino group). Acidic groups can be deprotonated using typical bases such as, for example, organic amines or alkali- or alkaline earth hydroxides.

Preferred zwitterionic or amphoteric polymers are:

copolymers formed from alkylacrylamide, alkylaminoalkyl methacrylate, and two or more monomers from acrylic acid and methacrylic acid as well as, if necessary, their esters, especially copolymers from octylacrylamide, acrylic acid, butylaminoethyl methacrylate, methyl methacrylate and hydroxypropyl methacrylate (INCI designation: Octylacrylamide/Acrylate/Butylaminoethyl Methacrylate Copolymer); copolymers, that are formed from at least one of a first type of monomer that possesses quaternary amino groups and at least one of a second type of monomer that possesses acid groups; copolymers from fatty alcohol acrylates, alkylamine oxide methacrylate and at least one monomer selected from acrylic acid and methacrylic acid as well as if necessary acrylic acid esters and methacrylic acid esters, especially copolymers from lauryl acrylate, stearyl acrylate, ethylamine oxide methacrylate and at least one monomer selected from acrylic acid and methacrylic acid as well as if necessary their esters; copolymers from methacryloyl ethyl betaine and at least one monomer selected from methacrylic acid and methacrylic acid esters; copolymers from acrylic acid, methyl acrylate and methacrylamidopropyltrimethylammonium chloride (INCI designation: Polyquaternium-47); copolymers from acrylamidopropyltrimethylammonium chloride and acrylates or copolymers from acrylamide, acrylamidopropyltrimethylammonium chloride, 2-amidopropylacrylamide sulfonate, and dimethylaminopropylamine (INCI designation: Polyquaternium-43); oligomers or polymers, producible from quaternary crotonoylbetaines or quaternary crotonoylbetaine esters.

In one embodiment, the agent of the present invention contains 0.01 to 15 wt. %, or preferably 0.5 to 10 wt. %, of at least one synthetic or natural nonionic film-forming polymer.

Suitable synthetic nonionic polymers are homo- or copolymers consisting of at least one of the following monomers: vinyl lactams such as, for example, vinyl pyrrolidone or vinyl caprolactam; vinyl esters such as, for example, vinyl acetate; vinyl alcohol, vinyl formamide, acrylamides, methacrylamides, alkyl acrylamides, dialkyl acrylamides, alkyl methacrylamides, dialkyl methacryl amides, alkyl acrylates, alkyl methacrylates, alkyl maleimides such as, for example, ethylmaleimide or hydroxyethylmaleimide, and alkylene glycols such as, for example, propylene or ethylene glycol, wherein the alkyl and/or alkylene groups of these monomers are preferably C1 to C7 alkyl groups, or especially preferably C1 to C3 alkyl groups.

Suitable homopolymers are, for example, those of vinylcaprolactam, vinylpyrrolidone or N-vinylformamide. Further suitable synthetic, nonionic polymers are, for example, polyacrylamides, polyethylene glycol/polypropylene glycol copolymers, copolymerides from vinylpyrrolidone and vinyl acetate, terpolymers from vinylpyrrolidone, vinyl acetate, and vinyl propionate, polyacrylamides; polyvinyl alcohols as well as polyethylene glycol/polypropylene glycol copolymers. Suitable natural film-forming polymers are, in particular, those based on saccharide, preferably glucans, e.g., cellulose and derivatives thereof. Suitable derivatives are, in particular, those with alkyl and/or hydroxyalkyl substituents, wherein the alkyl groups can have, for example, 1 to 20, or preferably 1 to 4 C atoms, e.g., hydroxyalkyl cellulose. Preferred nonionic polymers are: polyvinylpyrrolidone, polyvinylcaprolactam, vinylpyrrolidone/vinyl acetate copolymers, polyvinyl alcohol, isobutylene/ethylmaleimide/hydroxyethylmaleimide copolymer; copolymers from vinylpyrrolidone, vinyl acetate, and vinyl propionate.

In one embodiment, the agent according to the invention, as a hair-conditioning additive, contains at least one silicone compound preferably in a quantity of from 0.01 to 15 wt. %, or especially preferably of from 0.1 to 5 wt. %. The silicone compounds include volatile and nonvolatile silicones and silicones that are soluble and insoluble in the agent. One embodiment is high-molecular-weight silicone with a viscosity of 1,000 to 2,000,000 cSt at 25° C., or preferably 10,000 to 1,800,000 or 100,000 to 1,500,000. The silicone compounds include polyalkyl and polyaryl siloxanes, particularly with methyl, ethyl, propyl, phenyl, methylphenyl, and phenylmethyl groups. Polydimethyl siloxanes, polydiethyl siloxanes, and polymethylphenyl siloxanes are preferred. Also preferred are shine-providing, arylated silicones with a refractive index of at least 1.46 or at least 1.52. The silicone compounds include, in particular, the materials with the INCI designations Cyclomethicone, Dimethicone, Dimethiconol, Dimethicone Copolyol, Phenyl Trimethicone, Amodimethicone, Trimethylsilylamodimethicone, Stearyl Siloxysilicate, Polymethylsilsesquioxane, and Dimethicone Crosspolymer. Silicone resins and silicone elastomers are also suitable, wherein these are highly crosslinked siloxanes. Preferred silicones are: cyclic dimethyl siloxanes, linear polydimethyl siloxanes, block polymers from polydimethyl siloxane and polyethylene oxide and/or polypropylene oxide, polydimethyl siloxanes with terminal or lateral polyethylene oxide or polypropylenoxide radicals, polydimethyl siloxanes with terminal hydroxyl groups, phenyl-substituted polydimethyl siloxanes, silicone emulsions, silicone elastomers, silicone waxes, silicone gums, amino-substituted silicones, and silicones substituted with quaternary ammonia groups.

In one embodiment, the agent according to the invention contains a photoprotective material preferably in a quantity of from 0.01 to 10 wt. % or of from 0.1 to 5 wt. %, or especially preferably of from 0.2 to 2 wt. %. The photoprotective materials include, in particular, all the photoprotective materials mentioned in EP 1 084 696. The following are preferred: 4-methoxy cinnamic acid-2-ethylhexyl ester, methyl methoxy cinnamate, 2-hydroxy-4-methoxy benzophenone-5-sulfonic acid, and polyethoxylated p-aminobenzoate.

In one embodiment, the agent according to the present invention contains 0.01 to 20 wt. %, especially preferably 0.05 to 10 wt %, or very especially preferably 0.1 to 5 wt. % of at least one hair-conditioning additive, selected from betaine; panthenol; panthenyl ethyl ether; sorbitol; protein hydrolysates; plant extracts; A-B block copolymers from alkyl acrylates and alkyl methacrylates; A-B block copolymers from alkyl methacrylates, and acrylonitrile; A-B-A block copolymers from lactide and ethylene oxide; A-B-A block copolymers from caprolacton and ethylene oxide; A-B-C block copolymers from alkylene or alkadiene compounds, styrene and alkyl methacrylates; A-B-C block copolymers from acrylic acid, styrene, and alkyl methacrylates; star-shaped block copolymers; hyper-branched polymers; dendrimers; intrinsically electrically conducting 3,4-polyethylene dioxythiophenes and intrinsically electrically conducting polyanilines.

In one embodiment, the agent according to the invention contains 0.01 to 5 wt. %, or especially preferably 0.05 to 1 wt. %, of at least one preservative. Suitable preservatives are those materials listed with the “Preservatives” function in the International Cosmetic Ingredient Dictionary and Handbook, 10th edition, e.g., phenoxyethanol, benzylparaben, butylparaben, ethylparaben, isobutylparaben, isopropylparaben, methylparaben, propylparaben, iodopropynyl butylcarbamate, methyldibromoglutaronitrile, and DMDM hydantoin.

A particular embodiment of the invention relates to a hair-conditioning agent. Hair-conditioning agents are, for example, conditioners, treatments, hair-repair products, rinses, and the like. The hair-conditioning agent contains at least one hair-conditioning ingredient selected from the aforementioned silicone compounds, cationic or amine-substituted surfactants, and cationic or amine-substituted polymers. The hair-conditioning agent can be used in quantities of between 0.01 and 10.0 wt. %, or particularly between 0.01 and 5.0 wt. %, based on the finished product. The hair-conditioning agent according to the invention can, after application to the dry, damp, or wet hair, either remain in the hair or it can be rinsed out after a suitable action period. The action periods depend on the type of hair. As a general rule, action periods of between 0.5 and 30 minutes, particularly 0.5 and 10 minutes, and preferably between 1 and 5 minutes can be assumed.

In addition to the aforementioned cationic surfactants, other suitable cationic or amino-substituted surfactants are those of the formula R1-NH—(CH₂)n-NR2R3

or of the formula R1-NH—(CH₂)n-N⁺R2R3R4X⁻

wherein R1 is an acyl or an alkyl residue with 8 to 24 C atoms, which can be branched or linear, saturated or unsaturated, whereby the acyl and/or the alkyl residue can contain one or more OH groups, R2, R3 and R4 independently of one another are hydrogen, alkyl or alkoxyalkyl residues with 1 to 6 C atoms, which can be the same or different, saturated or unsaturated and can be substituted with one or more hydroxy groups, X⁻ is an anion, especially a halide ion or a compound of the general formula RSO₃—, wherein R has the meaning of saturated or unsaturated alkyl residues with 1 to 4 C atoms, and n means a whole number between 1 and 10, preferably from 2 to 5.

The active hair-conditioning compound is preferably an amidoamine and/or a quaternized amidoamine of the aforementioned formulae, wherein R1 is a branched or linear, saturated or unsaturated acyl residue with 8 to 24 C atoms that can contain at least one OH group. Preferred are such amines and/or quaternized amines, in which at least one of the residues R2, R3 and R4 means a residue according to the general formula CH₂CH₂OR5, wherein R5 can have the meaning of alkyl residues with 1 to 4 C atoms, hydroxyethyl or H. Suitable amines or amidoamines, which can be optionally quaternized, are especially such with the INCI names Ricinoleamidopropyl Betaine, Ricinoleamidopropyl Dimethylamine, Ricinoleamidopropyl Dimethyl Lactate, Ricinoleamidopropyl Ethyldimonium Ethosulfate, Ricinoleamidopropyltrimonium Chloride, Ricinoleamidopropyltrimonium Methosulfate, Cocamidopropyl Betaine, Cocamidopropyl Dimethylamine, Cocamidopropyl Ethyldimonium Ethosulfate, Cocamidopropyltrimonium Chloride, Behenamidopropyl Dimethylamine, Isostearylamidopropyl Dimethylamine, Stearylamidopropyl Dimethylamine, Quaternium-33, Undecyleneamidopropyltrimonium Methosulfate.

In a preferred embodiment, the agent according to the invention contains at least one pigment. The pigments can be colored pigments that provide coloring effects to the product mass or the hair, or they can be shine-enhancing pigments that provide shine effects to the product or the hair. The color or shine effects in the hair are preferably temporary, i.e., they remain until the next time the hair is washed and can be removed by washing the hair with typical shampoos. The pigments are not dissolved in the product mass and can be contained in a quantity of from 0.01 to 25 wt. %, with 5 to 15 wt. % being particularly preferred. The preferred particle size is 1 to 200 μm, particularly 3 to 150 μm, and especially preferably 10 to 100 μm. The pigments are practically insoluble colorants in the application medium and can be inorganic or organic. Inorganic-organic mixed pigments are also possible. Inorganic pigments are preferred. The advantage of inorganic pigments is their extraordinary resistance to light, weather, and temperature. The inorganic pigments can be of natural origin, for example, manufactured from chalk, ocher, umbra, green earth, burnt Terra di Siena, or graphite. The pigments can also be white pigments such as, for example, titanium dioxide or zinc oxide; black pigments such as, for example, iron oxide black; color pigments such as, for example, ultramarine or iron oxide red; shine pigments; metal effect pigments; pearl shine pigments; as well as fluorescence or phosphorescence pigments; wherein it is preferred if at least one pigment is a colored, nonwhite pigment. Metallic oxides, metallic hydroxides, and metallic oxide hydrates, mixed phase pigments, sulfur-containing silicates, metallic sulfides, complex metal cyanides, metallic sulfates, metallic chromates, and metallic molybdates, as well as the metals themselves (bronze pigments) are suitable. Titanium dioxide (CI 77891), black iron oxide (CI 77499), yellow iron oxide (CI 77492), red and brown iron oxide (CI 77491), manganese violet (CI 77742), ultramarine (sodium aluminum sulfosilicates, CI 77007, Pigment Blue 29), chromium oxide hydrate (CI77289), iron blue (ferric ferrocyanide, CI77510), and carmine (cochineal) are particularly suitable.

Pearl shine and color pigments based on mica and/or glimmer coated with a metallic oxide or a metallic oxychloride such as titanium dioxide or bismuth oxychloride as well as, if necessary, other color-providing materials such as iron oxides, iron blue, ultramarine, carmine, etc., and wherein the color can be determined by varying the layer thickness are especially preferred. These types of pigments are sold, for example, under the trade names Rona®, Colorona®, Dichrona®, and Timiron® by Merck, in Germany.

Organic pigments are, for example, the natural pigments sepia, Garcinia gummi-gutta, bone black, Van Dyke brown, indigo, chlorophyll, and other plant pigments. Synthetic organic pigments are, for example, azo-pigments, anthraquinoids, indigoids, and dioxazine, quinacridone, phthalocyanine, isoindolinone, perylene, perinone, metallic complex, alkali blue, and diketopyrrolopyrrol pigments.

In one embodiment, the agent according to the present invention contains 0.01 to 10 wt. %, or especially preferably 0.05 to 5 wt. %, of at least one particle-shaped material. Suitable materials are, for example, materials that are solid and in the form of particles at room temperature (25° C.). Silica, silicates, aluminates, alumina, mica, salts, particularly inorganic metallic salts, metallic oxides, e.g., titanium dioxide, minerals, and polymer particles are somewhat suitable. The particles are present in the agent in an undissolved, preferably steadily dispersed form and can be deposited on the hair in solid form after being applied to the hair and after the solvent has evaporated. A stable dispersion can be obtained by providing the composition with a yield point that is great enough to inhibit any sinking of the solid particles. A sufficient yield point can be obtained by using suitable gel-formers in a suitable quantity. Preferred particle-shaped materials are silica (silica gel, silicium dioxide) and metallic salts, particularly inorganic metallic salts, wherein silica is especially preferred. Metallic salts are, for example, alkaline or alkaline-earth halogenides such as sodium chloride or potassium chloride; and alkaline or alkaline earth sulfates such as sodium sulfate or magnesium sulfate.

An additional embodiment relates to an agent for permanently restructuring hair. It contains at least one reducing agent, particularly a keratin-reducing mercapto compound preferably in a quantity of from 0.5 to 15 wt. %. The permanent wave agent is preferably present as an aqueous, alkaline (pH=5 to 10) preparation, which contains e.g., cysteine, cysteamine, N-acetyl-L-cysteine, mercapto carboxylic acids such as, for example, mercaptoacetic acid or thiolactic acid, or salts of mercapto carboxylic acids such as, for example, ammonium and guanidine salts of mercaptoacetic acid or thiolactic acid as a keratin-reducing mercapto compound. The required alkalinity is obtained by adding ammonia, organic amines, ammonium and alkali carbonates, or bicarbonates. Neutral or acidic (pH=4.5 to 7) hair restructuring agents that have an effective content of sulfites or mercaptocarboxylic acid esters in an aqueous medium can also be considered. In the first case, preferably sodium or ammonium sulfite or the salt of sulfuric acid with an organic amine such as, for example, monoethanolamine and guanidine, can be used in a concentration of approximately 2 to 12 wt. % (calculated as SO2). In the latter case, mercaptoacetic acid mono glycol esters or glycerol esters are particularly used in a concentration of approximately 5 to 50 wt. % (corresponding to a content of 2 to 16 wt. % mercaptoacetic acid). The agent according to the invention for permanent restructuring of hair can also contain a mixture of the aforementioned keratin-reducing compounds. For the oxidative after-treatment, a fixing agent according to the invention containing at least one oxidizing agent can be used. Examples of oxidizing agents that can be used in one of these types of fixing agents are sodium and potassium bromate, sodium perborate, urea peroxide, and hydrogen peroxide. The concentration of oxidizing agent can be approximately 0.5 to 10 wt. %. Both the agent according to the invention for permanent hair restructuring as well as the fixing agent according to the invention can be present in the form of an emulsion or in thickened form on an aqueous basis, particularly as a cream, gel, or paste.

The composition to be used according to the invention can further contain any additive components that are conventional for hair treatment agents, for example perfume oils; opacifying agents such as, for example, ethylene glycol distearate, styrene/PVP copolymers or polystyrenes; humectants; shine providers; product dyes; antioxidants; each preferably in quantities of 0.01 to 10 wt. %, wherein the total quantity preferably does not exceed 10 wt. %.

The object of the invention is also a method for hair treatment, wherein
a product release system according to the invention is provided,
via the product release system, the composition contained therein is sprayed on the hair, and
the composition that is sprayed on is either rinsed out of the hair after an action period or it is left in the hair.

Instead of being sprayed directly onto the hair, the product can also be placed in the hands or on an application device such as, for example, a comb or a brush, and then distributed into the hair, particularly if the product has a snow-like consistency, or it is in the form of flakes or foam.

The products according to the invention are characterized, constrained by their special application with the special aerosol spray system to be used according to the invention, by an excellent distribution capacity in conjunction with a good hairstyle stability with good hold as well as shine for the hair. An additional advantage of the products according to the present invention is that differing spray properties can be precisely adjusted by simply varying the propellant, the propellant composition, or the propellant pressure; these spray properties were not previously possible for the underlying active ingredient compositions. The spray properties include everything from a fine aerosol atomized spray and snow-like drops to flakes of spray and spray foam.

The following examples should serve to illustrate further the object of the present invention.

### EXAMPLES

In the following examples, the individual active ingredient compositions were filled, along with the individually indicated propellants, into a pressure-resistant aerosol can and equipped with a capillary spray system, as can be obtained, for example, under the trade name TRUSPRAY® from Boehringer Ingelheim microParts GmbH.

### Example 1

### Solid Microemulsion

Active ingredient composition:

| Raw ingredients | Quantity |
|---|---|
| Low viscosity paraffin | 13.8 g |
| Oleth-10 | 12.5 g |
| OLETH-5 | 12.5 g |
| Polyquaternium-22 | 2.5 g |
| PEG-40 hydrogenated castor oil | 2.0 g |
| Perfume | 0.2 g |
| Dekaben LMB | 0.2 g |
| Water | balance to |
| | 100 g |

Consistency: Translucent, solid microemulsion

Filling with propellant:

| | 1-1 | 1-2 | 1-3 | 1-4 | 1-5 |
|---|---|---|---|---|---|
| Active ingredient composition | 50 g | 60 g | 70 g | 80 g | 40 g |
| Propane/butane 4.8 bar | 50 g | | 30 g | 10 g | 60 g |
| Propane/butane 2.7 bar | | 40 g | | | |
| DME | | | | 10 g | |

Spray properties:
1-1: Fine, dry aerosol spray
1-2: Fine, wet aerosol spray
1-3: Snow-like droplet formation
1-4: Spray foam
1-5: Very fine, dry aerosol

Practical tests were conducted on a mannequin wig. In a half-side comparison, the left side of the mannequin wig was treated with aerosol application 1-1 and the right side was treated with the propellant-free, highly viscous active ingredient composition. The aerosol version was easy to dispense and could be applied very precisely; it was also easier to distribute in the hair than the pure active ingredient. The hair treated with the aerosol application exhibited a significantly improved shine and significantly improved hold with the hairstyle in comparison to the hair treated with the pure active ingredient composition.

### Example 2

### Hair Styling Gel

Active ingredient composition:

| Raw ingredients | Quantity |
|---|---|
| Carbomer | 1.5 g |
| AMP | 1.35 g |
| Polyquaternium-11 | 1.0 g |
| PVP | 2.0 g |
| Glycerol | 1.5 g |
| PEG-40 hydrogenated castor oil | 1.0 g |
| Perfume | 0.2 g |
| Dekaben LMB | 0.2 g |
| Water | balance to |
| | 100 g |

Consistency: Highly viscous, clear gel

Filling with propellant:

| | 2-1 | 2-2 | 2-3 | 2-4 | 2-5 |
|---|---|---|---|---|---|
| Active ingredient composition | 50 g | 50 g | 50 g | 65 g | 40 g |
| Propane/butane 4.8 bar | 50 g | | | | |
| Propane/butane 2.7 bar | | 50 g | | | |
| DME | | | 50 g | 35 g | 60 g |

Spray properties:
2-1: Snow-like spray
2-2: Snow-like spray
2-3: Wet aerosol spray
2-4: Spray foam
2-5: Droplets (snow-like)

### Example 3

### Hair Styling Cream

Composition:

| Raw ingredients | Quantity |
|---|---|
| Carbomer | 1.00 g |
| AMP | 0.90 g |
| Polyquaternium-16 | 0.50 g |
| Polyvinylpyrrolidone | 3.00 g |
| Dimethicone | 15.00 g |
| PEG-40 hydrogenated castor oil | 0.20 g |
| Perfume | 0.20 g |
| Ethanol | 25.00 g |
| Water | balance to |
| | 100 g |

Consistency: Highly viscous cream

Filling with propellant:

| | 3-1 | 3-2 | 3-3 | 3-4 | 3-5 |
|---|---|---|---|---|---|
| Active ingredient composition | 50 g | 50 g | 50 g | 60 g | 70 g |
| Propane/butane 4.8 bar | 50 g | | | 40 g | 30 g |
| Propane/butane 2.7 bar | | 50 g | | | |
| DME | | | 50 g | | |

Spray properties:
2-1: Fine aerosol spray
2-2: Wet aerosol spray
2-3: Spray foam
2-4: Fine aerosol spray
2-5: Fine aerosol spray

### Example 4

### Hair Styling Wax

Composition:

| Raw ingredients | Quantity |
|---|---|
| Low viscosity paraffin | 40.00 g |
| Ceresin | 20.00 g |
| TRICETEARETH-4 PHOSPHATE | 10.00 g |
| PEG-25 HYDROGENATED CASTOR OIL | 2.50 g |
| Carnauba wax | 8.00 g |
| Beeswax | 8.00 g |
| Propylparabens | 0.20 g |
| Perfume | 0.30 g |
| Polyquaternium-4 | 2.50 g |
| Ethanol | 8.50 g |

### Consistency: Pasty wax

Filling with propellant:

| | 4-1 | 4-2 | 4-3 | 4-4 | 4-5 |
|---|---|---|---|---|---|
| Active ingredient composition | 50 g | 50 g | 50 g | 70 g | 60 g |
| Propane/butane 4.8 bar | 50 g | | | 30 g | 40 g |
| Propane/butane 2.7 bar | | 50 g | | | |
| DME | | | 50 g | | |

### Example 5

### Emulsion-Like, Creamy Hair-Repair Product/Treatment

Active ingredient composition:

| Raw ingredients | Quantity |
|---|---|
| Cetearyl alcohol | 10.00 g |
| Low viscosity paraffin | 4.00 g |
| Isopropyl myristate | 4.00 g |
| Polyquaternium-11 | 1.50 g |
| Cetyl trimethyl ammonium chloride | 1.50 g |
| Perfume | 0.40 g |
| Methylparaben | 0.30 g |
| Water | balance to |
| | 100 g |

Consistency: Thick cream

Propellant fillers:

| | 5-1 | 5-2 | 5-3 | 5-4 | 5-5 |
|---|---|---|---|---|---|
| Active ingredient composition | 50 g | 50 g | 50 g | 60 g | 70 g |
| Propane/butane 4.8 bar | 50 g | | | | |
| Propane/butane 2.7 bar | | 50 g | | | |
| DME | | | 50 g | 40 g | 30 g |

Spray properties:
5-1: Snow-like spray
5-2: Snow-like spray
5-3: Wet aerosol spray
5-4: Spray foam
5-5: Spray foam

### Example 6

### Hair Balsam

Composition:

| Raw ingredients | Quantity |
|---|---|
| Stearyl alcohol | 4.00 g |
| Cocamide MEA | 3.50 g |
| Polyquaternium-16 | 0.20 g |
| Stearyl trimethyl ammonium chloride | 1.50 g |
| Bisabolol | 0.10 g |
| Perfume | 0.20 g |
| PHB methyl ester | 0.20 g |
| Water | balance to |
| | 100 g |

Consistency: Viscous hair milk

Propellant fillers:

| | 6-1 | 6-2 | 6-3 | 6-4 | 6-5 |
|---|---|---|---|---|---|
| Active ingredient | 50 g | 50 g | 50 g | 60 g | 70 g |
| Propane/butane 4.8 bar | 50 g | | | | |
| Propane/butane 2.7 bar | | 50 g | | | |
| DME | | | 50 g | 40 g | 30 g |

Spray properties:
6-1: Snow-like spray
6-2: Snow-like spray
6-3: Wet aerosol spray
6-4: Spray foam
6-5: Spray foam

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A product release system for atomizing compositions comprising a pressure-resistant packaging, a spray head comprising a capillary, and a composition comprising a propellant, wherein the atomization occurs using said capillary and wherein said composition comprises at least one film-forming, hair-setting, or hair-conditioning polymer and said polymer is a cationic polymer.

2. A product release system according to claim 1, wherein said capillary has a diameter of 0.1 to 1 mm and a length of 5 to 100 mm.

3. A product release system according to claim 1, wherein the spray rate is 0.01 to 5 g/s.

4. A product release system according to claim 1, wherein the propellants are selected from the group consisting of propane, butane, dimethyl ether, fluorinated hydrocarbons, and mixtures thereof.

5. A product release system according to claim 1, wherein said cationic polymer is a homopolymer or a copolymer, said homopolymer or copolymer is constructed from at least one monomer selected from the group consisting of dialkylaminoalkyl acrylate, dialkylaminoalkyl methacrylate, monoalkylaminoalkyl acrylate, and monoalkyl aminoalkyl methacrylate, trialkyl methacryloxyalkyl ammonium, trialkyl acryloxyalkyl ammonium, dialkyl diallyl ammonium, and quaternary vinyl ammonium monomers with cyclic groups comprising cationic nitrogens.

6. A product release system according to claim 1, wherein said cationic polymer is selected from the group consisting of cationic cellulose compounds made from hydroxyethyl cellulose and diallyldimethyl ammonium chloride, cationic cellulose compounds made from hydroxyethyl cellulose and epoxide substituted with trimethyl ammonium, poly(dimethyldiallyl ammonium chloride), copolymers of acrylamide and dimethyldiallyl ammonium chloride, quaternary ammonium polymers, formed by the reaction of diethylsulfate with a copolymer of vinyl pyrrolidone and dimethylaminoethyl methacrylate, quaternary ammonium polymers of methylvinylimidazolium chloride and vinyl pyrrolidone, polyquarternium-35, polymers of trimethyl ammonium ethyl methacrylate chloride, polyquaternium-57, dimethylpolysiloxanes terminally substituted with quaternary ammonium groups, copolymers of vinyl pyrrolidone, dimethylaminopropyl methacrylamide and methacryloylamino propyl lauryl dimethyl ammonium chloride, chitosan, chitosan salts, hydroxyalkyl chitosan, hydroxyalkyl chitosan salts, alkyl hydroxyalkyl chitosans, alkyl hydroxyalkyl chitosan salts, N-hydroxyalkyl chitosan alkyl ether copolymers of vinyl caprolactam, vinyl pyrrolidone, and dimethylaminoethyl methacrylate, copolymers of vinyl pyrrolidone and dimethylaminoethyl methacrylate, vinyl caprolactam, dimethylaminopropylacrylamide, polyesters, formed from at least one monomer, which is a hydroxy acid that is substituted with at least one quaternary ammonium group, and oligoesters, formed from at least one monomer, which is a hydroxy acid that is substituted with at least one quaternary ammonium group.

7. A product release system according to claim 1, wherein the composition is a gel, wax, or emulsion.

8. A product release system according to claim 7, wherein the composition is a gel comprising at least one thickener or gel-former in a quantity of from 0.01 to 20 wt. % of the composition.

9. A product release system according to claim 8, wherein the thickener or gel-former is a thickening polymer, selected from the group consisting of copolymers which are made from at least one first type of monomer and from at least one second type of monomer, wherein said first type of monomer is selected from the group consisting of acrylic acid and methacrylic acid and wherein said second type of monomer is selected from the group consisting of ethoxylated fatty alcohol acrylic acid esters, crosslinked polyacrylic acid, crosslinked copolymers which are made from at least one first type of monomer, and from at least one second type of monomer, wherein said first type of monomer is selected from the group consisting of acrylic acid and methacrylic acid and wherein said second type of monomer is selected from the group consisting of C10 to C30 alcohol acrylic acid esters, copolymers made from at least one first type of monomer and from at least one second type of monomer, wherein said first type of monomer is selected from the group consisting of acrylic acid and methacrylic acid and wherein said at least one second type of monomer is selected from esters of itaconic acid and ethoxylated fatty alcohol, copolymers from at least one first type of monomer, from at least one second type of monomer and from at least one third type of monomer, wherein said first type of monomer is selected from the group consisting of acrylic acid and methacrylic acid and wherein said at least one second type of monomer is an ester of itaconic acid and ethoxylated C10 to C30 alcohol and wherein said third type of monomer is a C1 to C4 aminoalkyl acrylate, copolymers from two or more monomers selected from the group consisting of acrylic acid, methacrylic acid, acrylic acid esters, and methacrylic acid esters, copolymers of vinyl pyrrolidone and ammonium acryloyl dimethyltaurate, copolymers of ammonium acryloyl dimethyltaurate and at least one second monomer, wherein said second monomer is an ester of methacrylic acid and ethoxylated fatty alcohol, hydroxyethyl cellulose, hydroxypropyl cellulose, hydroxypropyl guar, glyceryl polyacrylate, glycerylpoly methacrylate, copolymers from at least one first type of monomer and styrene, wherein said first type of monomer is selected from the group consisting of C2 alkylene, C3 alkylene and C4 alkylene, polyurethanes, hydroxypropyl starch phosphate, polyacrylamide, copolymer of maleic acid anhydride and methyl vinyl ether, wherein said copolymer is crosslinked with decadiene, locust bean gum, guar gum, xanthan, dehydroxanthan, carrageenan, karaya gum, hydrolyzed corn starch, copolymers of polyethylene oxide, fatty alcohols, and saturated methylene diphenyl diisocyanate.

10. A product release system according to claim 7, wherein the composition is a waxy composition comprising at least one wax that is solid at 25° C. in a quantity of from 10 to 80 wt. %.

11. A product release system according to claim 10, wherein the wax is selected from the group consisting of paraffin waxes, polyolefin waxes, wool wax, wool wax alcohols, candelilla wax, olive wax, carnauba wax, Japan wax, apple wax, hydrogenated fats, fatty acid esters, fatty acid glycerides, fatty acid triglycerides, polyethylene glycol waxes, silicone waxes and mixtures thereof.

12. A product release system according to claim 7, wherein the composition is an emulsion-type composition selected from the group consisting of water-in-oil emulsions, oil-in-water emulsions and microemulsions, and wherein the composition comprises at least one emulsifier in a quantity of from 0.1 to 30 wt. %, and at least one oil in a quantity of from 1 to 20 wt. %, and water.

13. A product release system according to claim 12, wherein the oil is selected from the group consisting of silicone oils, mineral oils, isoparaffin oils, paraffin oils, squalane, sunflower seed oil, coconut oil, castor oil, lanolin oil, jojoba oil, corn oil, soy oil and mixtures thereof.

14. A product release system according to claim 12, wherein the emulsifier is selected from the group consisting of addition products of 2 to 30 mol ethylene oxide to C8 to C22 fatty alcohols, addition products of 1 to 5 mol propylene oxide to C8 to C22 fatty alcohols, addition products of 2 to 30 mol ethylene oxide and 1 to 5 mol propylene oxide to C8 to C22 fatty alcohols, addition products of 2 to 30 mol ethylene oxide to C12 to C22 fatty acids, addition products of 1 to 5 mol propylene oxide to C12 to C22 fatty acids, addition products of 2 to 30 mol ethylene oxide and 1 to 5 mol propylene oxide to C12 to C22 fatty acids, addition products of 2 to 30 mol ethylene oxide alkyl phenols with 8 to 15 C atoms in the alkyl group, addition products of 1 to 5 mol propylene oxide to alkyl phenols with 8 to 15 C atoms in the alkyl group, addition products of 2 to 30 mol ethylene oxide and 1 to 5 mol propylene oxide alkyl phenols with 8 to 15 C atoms in the alkyl group, C12 to C22 fatty acid monoesters of addition products of 1 to 30 mol ethylene oxide to glycerol, C12 to C22 fatty acid diesters of addition products of 1 to 30 mol ethylene oxide to glycerol, addition products of 5 to 60 mol ethylene oxide to castor oil, addition products of 5 to 60 mol ethylene oxide to hydrogenated castor oil, mono-, di-, or triesters of phosphoric acid with addition products of 2 to 30 mol ethylene oxide to C8 to C22 fatty alcohols, esters of saccharose with one or two C8 to C22 fatty acids, esters of sorbitan and one, two, or three C8 to C22 fatty acids and having a degree of ethoxylation of from 4 to 20, polyglyceryl fatty acid esters of one, two, or more C8 to C22 fatty acids with polyglycerol of 2 to 20 glyceryl units, alkylglycosides, C8-22 alkyldimethyl benzyl ammonium compounds, C8-22 alkyltrimethyl ammonium compounds, C8-22 alkyldimethyl hydroxyethyl ammonium compounds, di-(C8-22 alkyl)-dimethyl ammonium compounds, C8-22 alkylpyridinium salts, C8-22 alkylamido ethyl trimethyl ammonium ether sulfates, C8-22 alkylmethyl amine oxides, C8-22 alkyl amino ethyl dimethyl amine oxides, amidoamines, and quaternized amidoamines.

15. A product release system according to claim 1, wherein said composition comprises at least one additional active ingredient selected from the group consisting of hair-conditioning materials, hair-setting materials, silicone compounds, photoprotective materials, preservatives, pigments, direct-penetrating hair dyes, particle-shaped materials, oxidizing agents, reducing agents, oxidative hair dye precursor products, and mixtures thereof.

16. A product release system according to claim 15, wherein said active ingredients are contained in a quantity of from 0.01 to 20 wt. %.

17. A product release system according to claim 15, wherein a nonionic polymer is contained as said hair-conditioning material or as said hair-setting material, with said nonionic polymer being selected from the group consisting of polyvinylpyrrolidone, polyvinyl caprolactam, vinyl pyrrolidone/vinylacetate copolymers, polyvinylalcohol, isobutylene/ethylmaleimide/hydroxyethyl maleimide copolymer, copolymers from vinyl pyrrolidone, vinyl acetate, vinyl propionate, and mixtures thereof.

18. A product release system according to claim 15, wherein an anionic polymer is contained as said hair-conditioning material or as said hair-setting material, with said anionic polymer being selected from the group consisting of terpolymers from acrylic acid, ethyl acrylate, and N-tert-butylacrylamide, crosslinked or non-crosslinked vinyl acetate/crotonic acid copolymers, terpolymers from tert.-butylacrylate, ethyl acrylate, and methacrylic acid, sodium polystyrene sulfonate, copolymers from vinyl acetate, crotonic acid, and vinyl propionate, copolymers from vinyl acetate, crotonic acid, and vinyl neodecanoate, aminomethyl propanol acrylate copolymers, copolymers from vinyl pyrrolidone and at least one additional monomer selected from the group consisting of acrylic acid, methacrylic acid, acrylic acid esters, and methacrylic acid esters, copolymers from methyl vinyl ether and maleic acid monoalkyl esters, aminomethyl propanol salts of copolymers from allylmethacrylate and at least one additional monomer selected from the group consisting of acrylic acid, methacrylic acid, acrylic acid esters, and methacrylic acid esters, crosslinked copolymers from ethyl acrylate and methacrylic acid, copolymers from vinyl acetate, mono-n-butyl maleate, and isobornyl acrylate, copolymers from two or more monomers selected from acrylic acid, methacrylic acid, acrylic acid esters, and methacrylic acid esters, copolymers from octylacrylamide and at least one monomer selected from acrylic acid, methacrylic acid, acrylic acid esters, and methacrylic acid esters, and polyesters from diglycol, cyclohexanedimethanol, isophthalic acid, and sulfoisophthalic acid.

19. A product release system according to claim 15, wherein a zwitterionic or amphoteric polymer is contained as said hair-conditioning or as said hair-setting material, with said zwitterionic or amphoteric polymer being selected from the group consisting of copolymers from octylacrylamide, acrylic acid, butylaminoethyl methacrylate, methyl methacrylate, and hydroxypropyl methacrylate, copolymers from lauryl acrylate, stearyl acrylate, ethylamine oxide methacrylate, and at least one monomer selected from the group consisting of acrylic acid, methacrylic acid, acrylic acid esters, and methacrylic acid esters, copolymers from methacryloyl ethyl betaine and at least one monomer selected from the group consisting of methacrylic acid and methacrylic acid esters, copolymers from acrylic acid, methylacrylate, and methacrylamide propyl trimethylammonium chloride, oligomers or polymers that can be produced from monomers selected from the group consisting of quaternary crotonic betaines and quaternary crotonic betaine esters.

20. A product release system according to claim 15, comprising at least one silicone compound, which is selected from the group consisting of cyclic dimethylsiloxanes, linear polydimethylsiloxanes, block polymers from polydimethylsiloxane and at least one block selected from the group consisting of polyethylene oxide and polypropylene oxide, polydimethylsiloxanes with terminal or lateral residues selected from the group consisting of polyethylene oxide and polypropylene oxide, polydimethylsiloxanes with terminal hydroxyl groups, phenyl-substituted polydimethylsiloxanes, silicone emulsions, silicone elastomers, silicone waxes, silicone gums, amino-substituted silicones, silicones substituted with one or more quaternary ammonium groups, and mixtures thereof

21. A product release system according to claim 15, comprising at least one photoprotective material, which is selected from the group consisting of 4-methoxy cinnamic acid-2-ethylhexyl ester, methyl methoxy cinnamate, 2-hydroxy-4-methoxy benzophenone-5-sulfonic acid, polyethoxylated p-aminobenzoates, and mixtures thereof.

22. A product release system according to claim 15, wherein said active ingredient is selected from the group consisting of betaine, panthenol, panthenyl ethyl ether, sorbitol, protein hydrolysates, plant extracts, A-B block copolymers from alkyl acrylates and alkyl methacrylates, A-B block copolymers from alkyl methacrylates and acrylonitrile, A-B-A block copolymers from lactide and ethylene oxide, A-B-A block copolymers from caprolacton and ethylene oxide, A-B-C block copolymers from alkylene or alkadiene compounds, styrene and alkyl methacrylates, A-B-C block copolymers from acrylic acid, styrene, and alkyl methacrylates, star-shaped block copolymers, hyper-branched polymers, dendrimers, intrinsically electrically conducting 3,4-polyethylene dioxythiophenes, intrinsically electrically conducting polyanilines, and mixtures thereof.

23. A product release system according to claim 15, comprising at least one pigment, which is selected from the group consisting of titanium dioxide, black iron oxide, yellow iron oxide, red and brown iron oxide, manganese violet, ultramarine, chromium oxide hydrate, iron blue, bismuth oxichloride, carmine, mica based pearl shine pigments, mica based color pigments, wherein said mica based pearl shine pigments and said mica based color pigments are coated with a coating selected from the group consisting of metallic oxides and metallic oxychlorides and wherein the different colors of the mica based color pigments are produced by different layer thicknesses.

24. A product release system according to claim 15, wherein at least one particle-shaped material is contained, which is selected from the group consisting of silica, silicates, aluminates, alumina, mica, insoluble metallic salts, metallic oxides, minerals, and insoluble polymer particles.

25. The product release system according to claim 1, wherein said composition is a gel and comprises;
(a) 0.1 to 10 wt. % of at least one thickening polymer, selected from the group consisting of copolymers which are made from at least one first type of monomer and from at least one second type of monomer, wherein said first type of monomer is selected from the group consisting of acrylic acid and methacrylic acid and wherein said second type of monomer is an ethoxylated fatty alcohol acrylic acid ester, crosslinked polyacrylic acid, crosslinked copolymers which are made from at least one first type of monomer, and from at least one second type of monomer, wherein said first type of monomer is selected from the group consisting of acrylic acid and methacrylic acid and wherein said second type of monomer is a C10 to C30 alcohol acrylic acid ester, copolymers made from at least one first type of monomer and from at least one second type of monomer, wherein said first type of monomer is selected from the group consisting of acrylic acid and methacrylic acid and wherein said at least one second type of monomer is selected from esters of itaconic acid and ethoxylated fatty alcohol, copolymers from at least one first type of monomer, from at least one second type of monomer and from at least one third type of monomer, wherein said first type of monomer is selected from the group consisting of acrylic acid and methacrylic acid and wherein said at least one second type of monomer is an ester of itaconic acid and ethoxylated C10 to C30 alcohol and wherein said third type of monomer is a C1 to C4 aminoalkyl acrylate, copolymers from two or more monomers selected from the group consisting of acrylic acid, methacrylic acid, acrylic acid esters, and methacrylic acid esters, copolymers from vinyl pyrrolidone and ammonium acryloyl dimethyltaurate, copolymers from ammonium acryloyl dimethyltaurate and at least one second monomer, wherein said second monomer is an ester of methacrylic acid and ethoxylated fatty alcohol, hydroxyethyl cellulose, hydroxypropyl cellulose, hydroxypropyl guar, glyceryl polyacrylate, glycerylpoly methacrylate, copolymers from at least one first type of monomer and styrene, wherein said first type of monomer is selected from the group consisting of C2 alkylene, C3 alkylene and C4 alkylene, polyurethanes, hydroxypropyl starch phosphate, polyacrylamide, copolymer of maleic acid anhydride and methyl vinyl ether, wherein said copolymer is crosslinked with decadiene, locust bean gum, guar gum, xanthan, dehydroxanthan, carrageenan, karaya gum, hydrolyzed corn starch, copolymers from polyethylene oxide, fatty alcohols, and saturated methylene diphenyl diisocyanate; and
(b) 0.1 to 5 wt. % of at least one film-forming, hair-setting, or hair-conditioning cationic polymer, selected from the cationic polymers according to claim 6.

26. The product release system according to claim 1, wherein the composition is in the form of an oil-in-water emulsion, a water-in-oil emulsion, or a microemulsion and comprises;
(a) 1 to 20 wt. % of at least one oil or wax, selected from the group consisting of silicone oils, mineral oils, isoparaffin oils, paraffin oils, squalane, plant oils, paraffin waxes, polyolefin waxes, wool wax, wool wax alcohols, candelilla wax, olive wax, carnauba wax, Japan wax, apple wax, hydrogenated fats, fatty acid esters, fatty acid glycerides, fatty acid triglycerides, polyethylene glycol waxes, and silicone waxes;
(b) 0.01 to 30 wt. % of at least one emulsifier; and
(c) 0.01 to 20 wt. % of at least one film-forming, hair-setting, or hair-conditioning cationic polymer, selected from the cationic polymers according to claim 6.
